(19) United States
(12) Patent Application Publication
(10) Pub. No.: US 2026/0245655 A1
(43) Pub. Date: Aug. 20, 2026

(54) METHODS FOR DETERMINING DRUG CANDIDATES

(71) Applicant: MOSAIQUES DIAGNOSTICS AND THERAPEUTICS AG, Hannover (DE)

(72) Inventors: Marika MOKOU, Hannover (DE); Harald MISCHAK, Hannover (DE)

(21) Appl. No.: 19/162,108

(22) PCT Filed: Mar. 7, 2024
(86) PCT No.: PCT/EP2024/055936

§ 371 Date: Sep. 4, 2025

(30) Foreign Application Priority Data
Mar. 7, 2023 (EP) 23160357.2

(51) Int. Cl. G16B 20/00; A61K 31/15; A61K 31/343; A61K 31/4245; A61K 31/4706; A61K 31/4745; A61K 31/566; G16H 10/40; G16H 20/10

(52) U.S. Cl. CPC G16B 20/00; A61K 31/15; A61K 31/343; A61K 31/4245; A61K 31/4706; A61K 31/4745; A61K 31/566; G16H 10/40; G16H 20/10

(57) ABSTRACT

The present invention relates in a first aspect to a method for determining candidate molecules for treating a disease, disorder or condition associated with tissue alteration. In particular, the method refers to determining candidate molecules for treating bladder cancer in different stages of the disease, including an in silico prediction of drug response based on reversion of a disease specific molecular signature. For example, the CMap analysis is applied on data sets obtained based on proteomics and transcriptomics analyses. In a further aspect, the present invention relates to candidate molecules useful for treating said disease, disorder or condition at different stages of disease, in particular, allowing reversal of a more severe stage of disease to a more moderate stage or to stop progression to aggressive phenotypes like aggressive phenotype of bladder cancer. Finally, drug candidates useful in the treatment of said disease, in particular, the treatment of bladder cancer are provided.

The present invention relates in a first aspect to a method for determining candidate molecules for treating a disease, disorder or condition associated with tissue alteration. In particular, the method refers to determining candidate molecules for treating bladder cancer in different stages of the disease, including an in silico prediction of drug response based on reversion of a disease specific molecular signature. For example, the CMap analysis is applied on data sets obtained based on proteomics and transcriptomics.

In a further aspect, the present invention relates to candidate molecules useful for treating said disease, disorder or condition at different stages of disease, in particular, allowing reversal of a more severe stage of disease to a more moderate stage or to stop progression to aggressive phenotypes like aggressive phenotype of bladder cancer. Finally, drug candidates useful in the treatment of said disease, in particular, the treatment of bladder cancer are provided.

### PRIOR ART

Drug repurposing aims at the application of existing drugs for different purposes from their original context of use. This is a cost-effective strategy towards new therapies accelerating clinical translation in new applications. Namely, drug repurposing represents a cost-effective and efficient manner for new therapies against diseases, disorders and conditions not yet treated with said drugs. Due to the fact that the drugs have been already assessed for their safety and tolerability, drug repurposing shortens the drug development timelines, increases the odds of success, expedites regulatory approval and reduces the overall costs from bench to bedside. Namely, 30% of all drugs approved by the US Food and Drug Administration represent repurposed drugs. Strategies for repurposing, can be established by combining machine-learning compilation methods with chemical structures, genotypes, large-scale transcriptomics, proteomics data, e. g. Pushpakom S., et al., Nat. Rev.Drug. Discov., 2019, 18 (1): 41-58. Several resources have been introduced in the recent years for data-driven drug repurposing with the Connectivity Map (CMap) being one of the most well-known tools for signature matching drug repurposing. CMap was released in 2006 and is freely available via https://clue.io/. Through a pattern matching algorithm CMap establishes similarities among drugs based on gene expression profiles observed in disease affected cell lines, like cancer cell lines. Specifically, CMap relies on the signature reversion principle and allows researchers to interrogate their own gene list of interest, also called signature, against a large reference gene expression database that was generated by perturbing cell lines with a range of chemical compounds to identify drugs and new expression patterns similar (or opposite) to that of a query list. CMap analysis is typically based on data at the transcriptome level which normally constitutes a limitation of the approach when using as input other-omics, like proteomics, data. CMap has been applied for the identification of repurposed drugs in a wide range of diseases, based on single omics data.

Bladder cancer (BC) remains one of the most prevalent cancers globally, despite the significant progress that has been made in diagnosis, treatment and care in the recent years. BC is one of the diseases with highest lifetime treatment costs per patient. Typically, BC is histologically classified into Non-Muscle Invasive BC (NMIBC) and Muscle Invasive BC (MIBC). NMIBC accounts for approximately 75% of the newly diagnosed BC cases. Based on clinical pathological characteristics stratification of NMIBC to subgroups of low, intermediate and high risk of progression is applied. The standard of care for patients with low risk NMIBC is transurethral resection of the bladder tumor (TURBT) whereas for intermediate and high risk tumors TURBT should be followed by intravesical bacillus Calmette-Guérin (BCG) immunotherapy or instillations of chemotherapy for one to three years to reduce the frequency of recurrence and progression to MIBC. Nevertheless, almost 40% of the NMIBC patients fail to respond to first line BC therapy. The latter along with the high-recurrence rate of non-muscle invasive tumors (50% to 70%) and their progression to muscle invasive disease represent big challenges of current therapeutic approaches. Of the early diagnosed patients with BC, 25% will present with MIBC or metastatic disease. The gold standard of treatment for patients with muscle invasive non-metastatic BC remains cisplatin-based neoadjuvant (NAC) chemotherapy followed by radical cystectomy with lymph node dissection. Further, efficacy of immune checkpoint inhibitors (ICI) was recently reported and transformed the treatment landscape of advanced BC. Nevertheless, a high proportion of patients show low response rates and/or experience adverse events, which further necessitates improved patient stratification methods and better management of the observed ICI-related toxicity. Bladder disease management for MIBC as well as high risk NMIBC in cost-effective manners is urgently needed. In view of the high recurrence and the challenges of the current therapeutic schemes as well as acquisition of resistance and presence of numerous side-effects, it becomes evident that there is an urgent need for the identification of novel effective therapeutic strategies especially at earlier phases of BC, ideally before tumor has progressed to MIBC.

Recently CMap has been applied in the context of BC where a CMap based drug repurposing pipeline based on patients omic signature has been described, Mokou, M., et al, Cancers (Basel). 2020 Nov. 26; 12(12): 3519. However, the described pipeline was restricted to tissue proteomics data from patients with high-risk and low-risk Non-Muscle Invasive BC (NMIBC) but missed including information on the specific changes observed in tumor cells only (of note: the tumor cells only are the target for intervention), e.g. by including omics data from in vitro studies. Mokou M., et al., is not considering any proteomic changes of the bladder cancer cell lines and is only restricted to the tissue data of the two subtypes of NMIBC therein. No additional considerations and -omic data are used to filter out tissue data for those proteins that are not specifically expressed in the cancer cells and in the tumor niche, namely, a major drawback of the approach of this publication is that considered proteomic changes are not only changes of the cancer cells of the tumor but of the whole tumor niche. As a consequence, Mokou, M., et al. fails to provide novel therapeutic compounds for treatment of bladder cancer and, it was considered as unsuccessful requiring novel approaches.

### DESCRIPTION OF THE PRESENT INVENTION

The present inventors recognized that differences in stages and aggressiveness of a disease, disorder or condition can be characterized based on different-omic signatures using tissue proteomics, transcriptomics and omics data retrieved from various sources including tissue but also applying proteomics and transcriptomics data from available cell lines, like BC cell lines, allowing to determine candidate molecules, in particular, allowing repurposing of drugs for treating said disease, disorder or condition. By prediction of the drug response based on reversion of a disease specific molecular signature it is possible to determine suitable candidate molecules, in particular, allowing repurposing of the drugs accordingly. In particular, by combining the different signatures including the proteomics and transcriptomics data from cell lines, problems accruing in the past can be addressed. Namely, analysing tissue only whereby said tissue typically comprises multiple cell types on not only disease, disorder or conditions of specific cells, the data obtained may be falsified. This is particularly true when not only analysing the disease affecting cell or cell lines but even non cellular material present in the tissue is analysed including blood, plasma and extracellular matrix contributing to the final result. The present invention comprises the comparison of tissue data with data from specific cell lines restricting the information specifically obtained from the diseased cell. Particularly, in case of cancer cells and e.g. BC cells, the approach according to the present invention allows detecting credible candidate molecules to be tested as BC drugs accordingly. The molecular BC signatures restricted to features that are also present in BC cell lines were investigated in the examples through a drug repurposing pipeline employing the CMap tool resulting in the identification of candidate molecules with potential to reverse the disease signature.

Compared to the recent publication of the present inventors, see Mokou, M. et al, discussed above, the novel approach is based on restricting parameters used for prediction of potentially useful drugs to those proteins that are affected in the actual tumor cells. In contrast to the approach of the prior art, a clear distinction between the disease specific data and non-disease specific data can be obtained. The approach according to the present invention supports eliminating “bystander” from other cell types or also from the extracellular matrix, both of which are generally not available as targets for the disease directed intervention, like for the respective tumor directed intervention. In addition, the validity of the underlying data is enhanced according to the present invention by employing data from a second layer of molecular characteristics, the transcriptome, which is not considered in prior art. The approach according to the present invention enables collecting the specific modular changes with much higher confidence than the approach described in the literature, and in this way resulting in the prediction of potentially beneficial drugs with far superior success. For example, the prior art by Mokou, M. et al., does not consider any-omic changes of bladder cancer cell lines and is only restricted to the tissue data of the two types of NMIBC. In contrast, the -omic (proteomic and transcriptomic) differences according to the present invention extend throughout the whole spectrum of the disease. Further, according to the present invention, the results yield three different signatures developed based on tissue and cell line proteomics and transcriptomics data, which are further used in the CMap tool. Consequently, the method according to the present invention yields superior results compared to the prior art approaches exemplified by Mokou, M. et al.

That is, the present invention relates to a first aspect to a method for determining candidate molecules for treating a disease, disorder or condition associated with tissue alteration, like a tumor, in particular bladder cancer (BC) at different stages of said disease, disorder or condition comprising in silico prediction of drug response, based on reversion of a disease specific molecular signature, in particular, applying CMap analysis based on data obtained by
i) a first method of obtaining a disease, disorder or condition specific signature, like BC signatures from the altered tissue, like from BC tissue whereby a) these tissues are analyzed on proteomic differences associated with disease, disorder or condition associated progression, like tumor progression, in particular with BC progression and be detected in more than 10% of the analyzed tissue samples followed by b) identifying features characterizing different protein expression associated with the progression or the different stages of the disease, disorder or condition for providing a first data set for in silico prediction of drug response, in particular by CMap analysis;
ii) a second method of obtaining a disease, disorder or condition specific signature, like BC signatures whereby a) the cell proteome of cell lines, like tumor cell lines, in particular, BC cell lines depicting MIBC and NMIBC pathology, of different stages of progression of said disease, disorder or condition are analyzed and determining proteins demonstrating significant change in abundance between different stages of progression followed by b) comparing the protein of the cells, like the tumor cells, in particular, the BC, with the tissue proteome for providing a second data set for in silico prediction of drug response, in particular by CMap analysis;
iii) a third method of obtaining a third disease, disorder or condition specific signature, like a third BC signature comprising a) obtaining a cell lines transcriptomics data set whereby cell lines of different stages of the disease, disorder or condition are analysed; b) obtaining a cell line proteomics data set whereby cell lines of different stages of the disease, disorder or condition are analysed; c) comparing the proteomics data and transcriptomic data of a) and b) for obtaining features common in both data sets; and d) from the common features obtained in c) determining features being statistically significant different in at least one of the data set of a) and b) for providing a third data set for in silico prediction of drug response, in particular by CMap analysis; and 20
iv) the data set obtained in each of i), ii) and iii) are analyzed by a method for in silico prediction of drug response, in particular by CMap analysis for determining candidate molecules.

In this connection, the term “candidate molecules for treating a disease, disorder or condition associated with tissue alteration” refers to molecules or compounds that may have an effect on the disease, disorder or condition. For example, in case of tumors in particular, in case of BC, said candidate molecules represent compounds or molecules which demonstrate an effect on parameters associated with said tumor, like BC. In particular, according to the present invention it is possible that these candidate molecules display the activity on reversing more severe stages of the disease to less severe stages or do stop progression to aggressive phenotypes like aggressive phenotype of BC.

The terms compounds and molecules are used herein interchangeably unless otherwise indicated.

The term “reversion of a disease specific molecular signature” refers to the potential of reversing a specific expression profile of a given set of hallmark genes or proteins for a particular disease. The reversion includes embodiments with reversion from different stages in a disease, disorder or condition as well as reversion from a severe to a less severe situation within a single stage of the disease, disorder or condition. For example, in BC reversion may be from MIBC to NMIBC or within NMIBC from the severe subgroup to a moderate or low subgroup.

According to step i) of the method according to the present invention, tissues are analyzed to identify proteomic differences associated with the disease, disorder or condition and/or associated with progression of the same. For example, the progression includes the tumor progression or, for example in case of BC the progression from non-muscle-invasive bladder cancer (NMIBC) to muscle-invasive bladder cancer (MIBC), or in between the different stages of the NMIBC as identified above.

By detecting said proteomic differences resulting in identifying a number of proteins is present in more than 10% of the analyzed tissue samples, it is submitted that this is a trend of expression of the protein rather than a single event in one tissue sample. The first method according to step i) continued by identifying features characterizing different protein expression associated with a progression of the disease, disorder or condition. These features represent a first data set for the in silico prediction of drug response, like CMap analysis.

As used herein, the term “features” refers to molecular markers including RNA or proteins that further determine the characteristics of cells or tissues. Typically, the features derived from transcriptomic data are RNA or modification of nucleic acids, in particular, RNA while features of proteomics are peptides and proteins as well as modifications thereof.

Further, the first method may be exemplified to identify drug candidates having the potential to reverse the aggressive phenotype of BC and thus can be repurposed for BC treatment on the basis of different-omics signatures using tissue proteomics, transcriptomics and additional data (e.g. from data repositories, from literature, etc. which are publically available) and analyzing them in silico. The different data may be obtained from public sources including tissue data as well as proteomics and transcriptomics data from cell lines like BC cell lines. For example, the first method of step i) may be conducted as depicted in FIG. 1 as follows:

Raw proteomic data of 117 BC tissues were received from public sources Stroggilos, R., et al., Int. J. Cancer 2020, 146 (1): 281-294 and were further processed and evaluated focusing on the proteomic differences associated with BC progression, namely, differences between MIBC and NMIBC. The data were analyzed with three different analytical approaches [(Proteome Discoverer 1% FDR and no FDR threshold) and MaxQuant (1% FDR) given the complementarity of these approaches, generated data were compiled, while several stringency criteria were considered for shortlisting the differently abundant proteins including adjusted P-value using the Benjamini-Hochberg-method to adjust for multiple testing, frequency and fold change agreement among different analyses. As identified in step i) of the method according to the present invention, these proteins were detected in >10% of the samples with agreement in their trend of expression/abundance among the three different analytical approaches described above. These proteins were subsequently integrated with tissue transcriptomics and literature mined data from public sources, e.g. public databases or from literature; e.g. a search in the literature for previously published studies describing differences between MIBC and NMIBC at the RNA and protein level was done. Then all the data from the literature were integrated and this is defined as “literature mined data”. Further, the tissue transcriptomics data derived from the successful compilation of available transcriptomics/gene expression datasets from different repositories (cBioportal, GEO, ArrayExpress) Stroggilos R, et al. Cancers (Basel). 2022. 14 (10): 2542. As such, gene expression profiles from 1,054 primary BC tumor transcriptomes of treatment-naïve patients without any prior cancer history, along with profiles from 81 non-malignant urothelium tissues adjacent to the tumor site (NAU), generated using Affymetrix and Illumina platforms, were extracted. Data were processed, normalized, followed by the removal of batch effect using ComBat method and statistical analyses. Differentially expressed genes related to BC progression (MIBC vs NMIBC) were defined. Thus, the analysis and compilation of the data result in a multitude of features, in the present case 1575 features with different trend of expression between MIBC and NMIBC comprising the molecular signature of BC. This molecular signature of BC represents the first data set for the in silico prediction of drug response, in particular by CMap analysis.

According to step ii) the second method, see e.g. FIG. 1b), is applied whereby cell lines like tumor cell lines, in particular, BC cell lines, are analyzed depicting differences in progression, like depicting different stages of tumor progression with the goal of determining proteins demonstrating significant changes in expression between the different stages of progression, for example, with BC, the different stages are MIBC and NMIBC. The obtained proteins with difference in progression were compared with the tissue proteome.

The comparison of the tissue with the cell proteome reveals proteins having the same trend of expression when comparing MIBC with NMIBC accordingly. This additional signature of BC is used as the second data set for in silico prediction of drug response, in particular, by CMap analysis.

In step iii) of the method according to the present invention, see FIG. 1c), a third method is applied. Namely, this third method provides a third signature which is a data set allowing further in silico prediction of drug response, in particular, by CMap analysis. In this third method proteomics data and transcriptomics data of cell lines associated with the disease, disorder or condition are combined for obtaining features being statistically different comparing different progression states of the disease, disorder or condition for each data set of transcriptomics and proteomics data.

Namely, the cell lines transcriptomics data set is obtained whereby cell lines of different stages of the disease, disorder or condition are analysed. Further the cell line proteomics data set are obtained whereby cell lines of different stages of the disease, disorder or condition are analysed. Moreover, by comparing the proteomics data and transcriptomic data above, features common in both data sets are determined. Thereafter, among the common features obtained above, those being statistically significant different in at least one of the above transcriptomics and proteomics data set are determined providing a third data set for in silico prediction of drug response, in particular by CMap analysis.

The data sets obtained by each of the methods of step i), ii) and iii) are analyzed further in step iv) by a method of in silico prediction of drug response, in particular, by CMap analysis allowing to determine candidate molecules accordingly. In the example shown in FIG. 1, the analysis resulted in identifying a number of compounds with negative connectivity score. The identified compounds were further analysed on various aspects including toxicity, etc., and specific compounds were determined.

In an aspect, these candidate molecules are repurposed drugs.

In an embodiment of the present invention, the disease, disorder or condition is a tumor, like solid tumor. In an embodiment, the solid tumor is particularly BC. BC progression is differentiated between NMIBC and MIBC. In an aspect, the disease, disorder or condition is a tumor including solid tumors. The tumor may be bladder cancer or prostate cancer. In another aspect, the disease, disorder or condition is heart failure.

In an embodiment, candidate molecules are molecules being determined on their ability to allow reversal of an aggressive phenotype of the disease, disorder or condition associated with tissue alteration, like a tumor, in particular, BC to a less aggressive phenotype. For example, in case of BC the candidate molecule is considered to be able to inhibit the conservation to the MIBC scenario and reverse BC to less severe stage.

In an embodiment of the present invention, in step i) the proteins obtained from tissue proteomics are compared with the differently expressed genes relevant to stage progression from tissue transcriptomics. For example, the proteins obtained from the tissue proteomics showing differences in the progression of the disease are compared to tissue transcriptomics representing different stages of progression of said disease accordingly. For example, in case of BC the stages include NMIBC and MIBC accordingly.

In an embodiment of the present invention, the cell lines analyzed in the second method as identified in step ii) are cell lines having either NMIBC or MIBC phenotypes. The cell lines that were analyzed included the HBLAK and RT112 cells which were both annotated as derived from patients with NMIBC as well as the BFTC-905, HT1376 and T24 cells that were all annotated as derived from patients with MIBC.

Moreover, in an embodiment of the present invention the method is characterized in that proteins determined in step ii) include among others proteins for which disease specificity in the analysed tissue, like tumor association is known.

Further, according to an embodiment of the present invention, the tissue proteome used in the method according to step ii) is the tissue proteome of step i). That is, an identical tissue proteome is used.

In another embodiment, the transcriptomics used in step iii) is a transcriptomics from cell lines from urinary tract when looking for candidate molecules of treating BC.

In an embodiment, the in silico step of predicting drug response based on the data sets obtained in each of the steps i), ii) and iii) is by CMap analysis. For example, the candidate molecules are determined by a negative enrichment score during CMap analysis. The skilled person is well aware of methods to identify the same and determining negative enrichment scores accordingly.

In an embodiment, the method includes furthermore the step of determining data of treating a disease, disorder or condition, associated with tissue alteration, like a tumor, in particular, BC of the candidate molecules identified. in particular, data present in database or otherwise publicly available of the candidate molecules in treating a different disease, like in treating cancer, in particular, cancer other than BC or known candidate molecules in treating different (other than cancer) diseases. In an aspect, the method includes the step of determining data of treating the tumor whereby data present in database or otherwise publically available of the candidate molecules in treating a tumor different to the one the candidate molecules are determined, are uncovered. For example, in an embodiment, the data are data for treating cancer other than BC.

The method according to the present invention allows identifying suitable candidate molecules for further testing of effectiveness and efficacy in treating the respective disease, disorder or condition. In particular, repurposing of known drugs described for treating different diseases, disorders or conditions, in particular, non-related diseases, disorders or conditions, are identified and determined according to the present invention.

In a further aspect, the present invention relates to the candidate molecule obtainable by a method according to the present invention. That is, in a further aspect, the present invention relates to a candidate molecule not described for treating a disease, disorder or condition associate with tissue alteration, like a tumor, in particular, bladder cancer at different stages of said disease, disorder or condition obtained by a method according to the present invention for use in treating a disease, disorder or condition associated with tissue alteration, like a tumor, in particular bladder cancer at different stages of said disease, disorder or condition.

For example, the candidate molecule is cephaeline.

In another embodiment, the drug candidate is a compound of general formula II

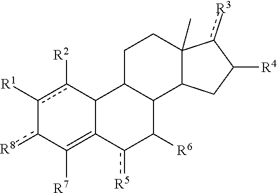

wherein
R1, R2, R4, R6 and R7 each are independently selected from H, C1-C4 alkyl, substituted C1-C4 alkyl, halogen;
R3 and R8 are each individually selected from O, OH, O C1-C4 alkyl;
----- is a single or double bond;
R5 is a compound of CHn(C1-C4) m with n and m being an integer of 0 to 3 and n+m is 3 when ----- is a single bond, and
R5 is CH2, CH C1-C4 alkyl or C (C1-C4 alkyl)2 when ----- is a double bond, for use in the treatment of BC.

In an embodiment, the compound of formula II is exemestane.

In addition, according to the present invention, other favorable candidate molecules include compounds from the group of fluvoxamine or a derivative thereof; amiodarone or a derivative thereof, isradipine or a derivative thereof, or amodiaquine or a derivative thereof.

In a preferred embodiment, the candidate molecule in the treatment of BC is cephaeline, in another embodiment, the candidate molecule is exemestane.

The term “derivative thereof” refers to compounds which can be synthesized by chemical transformations of the compounds functional groups using standard reactions.

In addition, unless otherwise indicated, the candidate compounds may be present in the form of a prodrug, metabolite or derivative. Further, the candidate compounds may be present in the form of pharmaceutically acceptable salts or hydrates.

Based on the candidate molecules determined in the method of the present invention further selection of possible candidate molecules is possible. For example, the candidate molecules are searched for known information on biological effectiveness and, in particular, any disease association. This allows focusing on suitable compounds as candidate molecules for drugs.

The candidate molecules or drug candidates are screened for their efficacy on BC cells or tumors. For example, in the present case, a list of twenty four compounds was identified as given below.

TABLE 1
The list of the 24 compounds which were
shortlisted for in vitro screening.

| Name of the compound | Class |
|---|---|
| ursodeoxycholic acid | a secondary bile acid |
| (synonym: ursodiol) | |
| cephaeline | alkaloid with emetic properties |
| sulfamethoxypyridazine | a sulfonamide antibacterial |
| guanethidine | antihypertensive |
| exemestane | aromatase inhibitor |
| | member of the class of antiestrogens |
| DL-thiorphan | enkephalinase inhibitor |
| 1,4-chrysenequinone | activator of aryl hydrocarbon receptor (AhR) |
| fenspiride | Antiasthmatic & COPD Preparations |
| morantel | anthelmintic drug |
| latamoxef | Antibacterial agents |
| flavoxate | anticholinergic with antimuscarinic effects |
| propantheline bromide | anticholinergic with antimuscarinic effects |
| tubocurarine chloride | neuromuscular blocking agents |
| metolazone | diuretics |
| sulfanilamide | anti-infective |
| demeclocycline | antibiotic |
| methylergometrine | ergot alkaloids |
| bupropion | antidepressants |
| fluvoxamine | selective serotonin reuptake inhibitors |
| | (SSRIs) |
| amiodarone | antiarrhythmics |
| primidone | anticonvulsants |
| iobenguane | Antineoplastic Agents - Radiopharmaceutical |
| amodiaquine | antimalarial drug |
| isradipine | Antihypertensive |
| | calcium channel blockers |

These twenty four compounds were selected based on the list of candidate molecules. Some of these drug candidates have been previously used in other cancer types, further supporting the potential to provide a positive result during the studies in BC. The compounds shown in Table 1 belong to different drug classes as outlined.

The impact of the drug candidates onto the malignant phenotype of BC cells was assessed in vitro in a panel of multi-origin BC cell lines representing different stages during disease progression. These included non-muscle invasive (RT112) and muscle invasive (T24, BFTC905, HT1376) cells.

Of the twenty-four compounds, cephaeline, exemestane, 1,4-chrysenequinone, amodiaquine, isradipine, amiodarone and fluvoxamine prevented cell growth at relatively low concentrations. Accordingly, these compounds were selected for further investigation, here providing the response curves for four different BC cell lines. Representative response curves of the seven compounds are presented in FIG. 2, along with the respective IC50s. A clear impact was observed on the proliferation of the BC cells for all seven compounds.

That is, the seven drugs significantly decreased the growth rate of the BC cells in vitro.

In the next step, the impact of the seven compounds is investigated in vivo in xenograft mouse models.

In vivo testing of exemestane, cephaeline and 1,4 chrysenequinone was performed in xenograft mouse models. Particularly, the model was established after injecting 4*106 BFTC905 cells subcutaneously into the tail base of NOD/SCID male mice. Male mice were selected for these experiments since males are more prone to develop BC. The treatments started three days after the injections of the cancer cells. The treatment scheme was 30 mg/kg/every 3rd day for exemestane, 5 mg/kg/daily for cephaeline and 5 mg/kg/daily for 1,4 chrysenequinone. As controls were used animals injected with DMSO diluted in PBS (at a final concentration similar to the one of the compounds). As shown in FIG. 3, a clear impact on tumor establishment or growth was observed for cephaeline and exemestane respectively. The tumor volume of the animals treated with 1,4 chrysenequinone was similar to the one of the control-DMSO group, indicating lack of efficacy of this drug in vivo. The impact of the drugs was observed at least for 17 days until the tumor volume of the control animals reached >300 mm³.

Based on the above, the present inventors identified cephaeline, exemestane as suitable drug candidates or candidate molecules for treating BC. In particular, it is submitted that these compounds are suitable for reversal of an aggressive phenotype of BC to a less aggressive phenotype for inhibiting progression to a more severe phenotype accordingly.

Moreover, by applying the method according to the present invention, the inventors recognized further compounds representing suitable candidate molecules. Said compounds are selected from the group of
i) Fluvoxamine or a derivative thereof;
ii) Amiodarone or a derivative thereof;
iii) Isradipine or a derivative thereof; or
iv) Amodiaquine or a derivative thereof.

Suitability of the method according to the present invention is demonstrated further by way of analyzing prostate cancer (PC). Namely:

Raw proteomic data of a total of 104 data sets were analysed including 40 previously published proteomics datasets from fresh frozen tissue samples from benign and malignant prostatic tissue [1], paraffin embedded tissue samples (FFPE) [2] and 64 publicly available datasets retrieved from MassIVE Repository [3]. The data were evaluated focusing on the proteomic differences between benign and malignant prostatic tissue. The data were analyzed with three different analytical approaches [(Proteome Discoverer 1% FDR and no FDR threshold) and MaxQuant (1% FDR). Generated data were compiled and differently abundant proteins were identified applying adjusted P-value using the Benjamini-Hochberg-method to adjust for multiple testing. As identified in step i) of the method, only proteins detected in >10% of the samples with agreement in their trend of expression/abundance among the three different analytical approaches described above were included. The Spearman's rank-order correlation was employed to identify proteins associated with PCa progression. For the latter, the protein abundance was correlated with ISUP grading system GG (grade group) from patients with PCa. As identified in step i) of the method according to the present invention, these proteins were detected in >10% of the samples with agreement in their trend of expression/abundance among the three different analytical approaches described above, resulting in 2802 significant proteins. These proteins were subsequently integrated with tissue transcriptomics (3696 significant genes) and literature mined data (371 features) from previously published studies describing differences between benign and malignant prostatic tissue at the RNA and protein level, subsequently termed “literature mined data”. In detail, tissue transcriptomics data were retrieved from Prostate Cancer Transcriptome Atlas (http://www.thepcta.org/) [4]. Specifically, a dataset comprised of 18,390 human genes with expression data for 2,115 tissue samples (1321 malignant and 749 normal/benign samples) was retrieved. For the subsequent analysis only samples originating from prostate tissue (either benign or malignant) and for which information on the Gleason score was available (in the case of malignant samples) were included. 12,862 differentially expressed genes were found to be significantly correlated with PCa progression (malignant versus benign). Of those 3696 significant genes features were significantly correlated with different Gleason grades of prostate aggressiveness. After compilation of the three abovementioned inputs (2802 sig. proteins, 3696 sig. genes and 371 literature features), 1976 features were considered as commonly significant with same directionality. These 1976 features comprise the signature of PCa. This molecular signature of PCa represents the first data set for the in silico prediction of drug response, in particular by CMap analysis.

According to step ii) the second method (as outlined in FIG. 1b for BC), is applied whereby cell lines like tumor cell lines, in particular, PCa cell lines specifically LNCaP, PC3 and DU145, are analyzed depicting differences in progression, like depicting different stages of tumor progression with the goal of determining proteins demonstrating significant changes in expression between the different stages of progression. In detail, DU145 and PC3 are castration resistant cell lines while LNCaP are characterized as AR sensitive cells (representing a less advances disease stage). The obtained proteins with significant abundance differences related with disease progression (cell proteome: 3086) were compared with the tissue proteome (tissue proteome: 2802).

The comparison of the tissue with the cell proteome revealed 538 proteins having the same trend of expression in significantly depicting PCa progression. This additional signature of PCa is used as the second data set for in silico prediction of drug response, in particular, by CMap analysis.

In step iii) of the method according to the present invention (as outlined in FIG. 1c for BC), a third method is applied. Namely, this third method provides a third signature which is a data set allowing further in silico prediction of drug response, in particular, by CMap analysis.

In this third method proteomics data and transcriptomics data of cell lines associated with the disease, disorder or condition are combined for obtaining features being statistically different comparing different progression states of the disease, disorder or condition for each data set of transcriptomics and proteomics data.

Namely, the cell lines transcriptomics data set is obtained whereby cell lines of different stages of the disease, disorder or condition are analysed. In case of PCa, LNCaP, DU145 and PC3 were considered, as depicting different stages of PCa, as described in step ii) and considering that both transcriptomics and proteomics datasets exist. By integrating the proteomics data (3056 significant proteins) and transcriptomic data (4,410) above, 1015 commonly significant features in both data sets are determined. Thereafter, among the common features obtained above, those being statistically significant different in at least one with the same trend of expression, resulted in 246 significant features, providing a third data set for in silico prediction of drug response, in particular by CMap analysis.

The data sets obtained by each of the methods of step i), ii) and iii) are analyzed further in step iv) by a method of in silico prediction of drug response, in particular, by CMap analysis allowing to determine candidate molecules accordingly.

In an embodiment, candidate molecules are molecules being determined on their ability to allow reversal of an aggressive phenotype of the disease, disorder or condition associated with tissue alteration, like a tumor, in particular, PCa to a less aggressive phenotype. For example, in case of PCa the candidate molecule is considered to be able to inhibit the progression or conversion to the malignant scenario and reverse PCa to less severe stage, represented by the ability to respond to hormonal treatment.

In an embodiment of the present invention, in step i) the proteins obtained from tissue proteomics are compared with the differently expressed genes relevant to PCa progression from tissue transcriptomics. For example, the proteins obtained from the tissue proteomics showing differences in the progression of the disease are compared to tissue transcriptomics representing different stages of progression of said disease accordingly. For example, in case of PCa the stages include malignant versus benign as well as the different Gleason grades.

In an embodiment of the present invention, the cell lines analyzed in the second method as identified in step ii) are cell lines having either malignant or benign phenotypes. The cell lines that were analyzed included the DU145, LNCaP, and PC3 that were all annotated as either hormone sensitive or castration resistant cell lines.

Moreover, in an embodiment of the present invention the method is characterized in that proteins determined in step ii) include among others proteins for which disease specificity in the analysed tissue, like tumor association is known.

Further, according to an embodiment of the present invention, the tissue proteome used in the method according to step ii) is the tissue proteome of step i). That is, an identical tissue proteome is used.

In another embodiment, the transcriptomics dataset used in step iii) is a transcriptome from cell lines from prostate when searching for candidate molecules of treating PCa.

In an embodiment, the in silico step of predicting drug response based on the data sets obtained in each of the steps i), ii) and iii) is by CMap analysis. In this example, the candidate molecules are determined by a negative enrichment score during CMap analysis. The skilled person is well aware of methods to identify the same and determining negative enrichment scores accordingly.

In an embodiment, the method includes furthermore the step of determining data of treating a disease, disorder or condition, associated with tissue alteration, like a tumor, in particular, PCa of the candidate molecules identified.

Based on the candidate molecules determined in the method of the present invention (steps i, ii, and iii), 37 of 43 drugs that are approved by the Food and Drug Administration (FDA) were predicted to revert the tumor phenotype, further demonstrating the validity of the approach (listed in Table 1).

Among others, in the predicted list (Table 2), there are multiple androgen agonists and antagonists, PARP inhibitors, CYP17A1 inhibitors (abiraterone), cabazitaxel/docetaxel (chemotherapy), mitoxantrone (topoisomerase inhibitor) and even several 5-alpha reductase inhibitors (dutasteride and finasteride) which are primarily used to treat BPH.

Among the 6 that were not identified (Table 2), four were radiopharmaceuticals and two were referring to autologous immunotherapy, where patient-derived T cells are boosted to produce immune response against tumour in an ex vivo setting are then injected back to the patients. Thus, based on the underlaying mechanism of action, these six were not expected to be in the list of molecular-targeting compounds.

TABLE 2
List of cancer drugs approved by the Food and Drug
Administration (FDA) for prostate cancer. The
list includes generic names and brand names.

| | CMap | Mechanism |
|---|---|---|
| Drugs Approved for Prostate Cancer | prediction | of Action |
| Abiraterone Acetate | Identified | CYP17A1i |
| Yonsa (Abiraterone Acetate) | Identified | CYP17A1i |
| Zytiga (Abiraterone Acetate) | Identified | CYP17A1i |
| Docetaxel | Identified | Chemotherapy, mitotic |
| | | inhibitor |
| Talazoparib Tosylate | Identified | PARPi |
| Talzenna (Talazoparib Tosylate) | Identified | PARPi |
| Xofigo (Radium 223 Dichloride) | Not | Radiopharmaceutical |
| | Identified | |
| Xtandi (Enzalutamide) | Identified | AR antagonist |
| Bicalutamide | Identified | AR antagonist |
| Cabazitaxel | Identified | Taxane, microtubule |
| | | inhibitor |
| Casodex (Bicalutamide) | Identified | AR antagonist |
| Enzalutamide | Identified | AR antagonist |
| Flutamide | Identified | AR antagonist |
| Goserelin Acetate | Identified | GnRHR agonist |
| Jevtana (Cabazitaxel) | Identified | Taxane, microtubule |
| | | inhibitor |
| Lynparza (Olaparib) | Identified | PARPi |
| Mitoxantrone Hydrochloride | Identified | Topoisomerase |
| | | inhibitor |
| Nilandron (Nilutamide) | Identified | AR antagonist |
| Nilutamide | Identified | AR antagonist |
| Olaparib | Identified | PARPi |
| Relugolix | Identified | GnRHR antagonist |
| Rubraca (Rucaparib Camsylate) | Identified | PARPi |
| Rucaparib Camsylate | Identified | PARPi |
| Taxotere (Docetaxel) | Identified | Chemotherapy, mitotic |
| | | inhibitor |
| Zoladex (Goserelin Acetate) | Identified | GnRHR agonist |
| Provenge (Sipuleucel-T) | Not | vaccine (autologous |
| | Identified | immunotherapy) |
| Sipuleucel-T | Not | vaccine (autologous |
| | Identified | immunotherapy) |
| Lutetium Lu 177 Vipivotide | Not | radiopharmaceutical |
| Tetraxetan | Identified | |
| Pluvicto (Lutetium Lu 177 Vipivotide | Not | radiopharmaceutical |
| Tetraxetan) | Identified | |
| Radium 223 Dichloride | Not | radiopharmaceutical |
| | Identified | |
| Akeega (Niraparib Tosylate | Identified | PARPi |
| Monohydrate and Abiraterone | | |
| Acetate) | | |
| Darolutamide | Identified | PARPi |
| Niraparib Tosylate Monohydrate and | Identified | PARPi |
| Abiraterone Acetate | | |
| Apalutamide | Identified | AR antagonist |
| Erleada (Apalutamide) | Identified | AR antagonist |
| Nubeqa (Darolutamide) | Identified | AR antagonist |
| Degarelix | Identified | GnRHR antagonist |
| Firmagon (Degarelix) | Identified | GnRHR antagonist |
| Orgovyx (Relugolix) | Identified | GnRHR antagonist |
| Eligard (Leuprolide Acetate) | Identified | GnRHR agonist |
| Leuprolide Acetate | Identified | GnRHR agonist |
| Lupron Depot (Leuprolide Acetate) | Identified | GnRHR agonist |

### REFERENCES

1. Latosinska, A., et al., Molecular Changes in Tissue Proteome during Prostate Cancer Development: Proof-of-Principle Investigation. Diagnostics (Basel), 2020. 10(9).
2. Lygirou, V., et al., Proteomic Analysis of Prostate Cancer FFPE Samples Reveals Markers of Disease Progression and Aggressiveness. Cancers (Basel), 2022. 14(15).
3. Sinha, A., et al., The Proteogenomic Landscape of Curable Prostate Cancer. Cancer Cell, 2019. 35(3): p. 414-427 e6.
4. You, S., et al., Integrated Classification of Prostate Cancer Reveals a Novel Luminal Subtype with Poor Outcome. Cancer Res, 2016. 76(17): p. 4948-58.
5. Lygirou, V., et al., Plasma proteomic analysis reveals altered protein abundances in cardiovascular disease. J Transl Med, 2018. 16(1): p. 104.
6. Schanstra, J. P., et al., Systems biology identifies cytosolic PLA2 as a target in vascular calcification treatment. JCI Insight, 2019. 4(10).
7. Jager, M., et al., Intrasurgical Protein Layer on Titanium Arthroplasty Explants: From the Big Twelve to the Implant Proteome. Proteomics Clin Appl, 2019. 13(2): p. e1800168.

Moreover, the method is applied for the disease heart failure shown in the following:

LC-MS/MS analysis was performed for a total of 46 heart tissue samples that were further processed and evaluated focusing on the proteomic differences associated with heart failure (HF). Biopsy tissue samples were obtained from explanted failing hearts (cases) (n=34) and donor hearts discarded from implantation (controls) (n=12). Cases included patients with dilated cardiomyopathy (n=14), ischaemic cardiomyopathy (n=15) and hypertrophic cardiomyopathy (n=5). 184 proteins were found to be potentially significantly associated with HF (BH adjusted p-value<0.05), detected in >60% of the samples (cases or controls). These proteins were subsequently integrated with tissue transcriptomics and literature mined data from public sources.

Tissue transcriptomics data were retrieved from publicly available repositories, including Expression Omnibus (GEO) (Edgar R et al. Nucleic Acids Res. 2002; 30(1): 207-10) and Array Express (Parkinson H et al. Nucleic Acids Res. 2007; 35 (Database issue): D747-50) using the following keywords: (heart AND fibrosis), (heart failure), (cardiac AND fibrosis), (heart failure AND fibrosis), (cardiomyopathy). Only studies from the last 10 years analyzing human heart samples using an array platform (Affymetrix and Illumina platforms) and relevant in the context of HF were considered. Further inclusion criteria included information on etiology (ischemic, non-ischemic, or hypertrophic), studies in adults, and left ventricle sampling. Available metadata were retrieved. Based on these criteria, 362 datasets from patients with HF and 193 controls were included. The analysis followed the previously established pipeline (Stroggilos et al., Cancers (Basel). 2022; 14(10): 2542). All analyses were performed using R statistical software. The batch effect was removed using the ComBat algorithm. Statistical analysis between all HF cases and controls was performed based on the ComBat corrected data (pooled analysis) and individual datasets (6 datasets). 306 features with differential expression between HF and non-HF groups in pooled analysis and in at least 3 individual datasets with a consistent regulation trend across all datasets were shortlisted.

In a third step literature was investigated and 391 features reported changed in HF were extracted. Combining all three datasets a signature comprised of a total of 797 features was defined.

Since cell lines representing different stages of the disease (heart failure) are not available. Previously published proteomics (PMID: 36078153, 2785 identifications), and transcriptomic (PMID: 34959166, 12237 transcripts consistently detected across all cell line replicates) data from cardiomyocytes were investigated to restrict the analysis to proteins present in those cells.

Comparison of the signature comprised of 797 features defined above with the proteins and transcripts detected in cardiomyocytes resulted in the definition of 167 proteins of cellular (cardiomyocyte) origin, as supported by both proteomics and transcriptomic data, predicted to be changed in HF. This signature, in this example consisting of 167 features (proteins), is a data set allowing further in silico prediction of impact of specific drug, in particular, by CMap analysis. The skilled person is well aware of methods to identify the same and determining negative enrichment scores accordingly.

In an embodiment, the method includes furthermore the step of collecting data on the potential therapeutic impact of the candidate molecules in the context of HF (in this example). Based on the candidate molecules determined in the method of the present invention, there were multiple drugs/compounds previously reported about their potency in HF treatment, thereby further supporting the validity of the approach. Among the top ranked (connectivity score>0.8) were included: Auranofin (PMID: 29554646), Parthenolide (PMID: 21223972), THZ-1 (PMID: 35896549), Digoxin (PMID: 35371861), Alvespimycin (PMID: 32979174), Lomitapide (PMID: 28925748), Vorinostat (PMID: 33970677, PMID: 36098145), Sulforaphane (PMID: 26583056), Ouabain (PMID: 26587223), manumycin-a (PMID: 17363690) and bufalin (PMID: 37243403).

### BRIEF DESCRIPTION OF THE FIGURES

FIG. 1. Schematic representation of the workflow followed for the generation of BC signatures that were then used for the identification of drugs that have the potential to reverse the aggressiveness of BC. a) Combination of tissue proteomics, transcriptomics and tissue data (RNA and protein expression data) retrieved from the literature; b) Combination of BC cell lines proteome with tissue proteome; c) Compilation of BC cell lines transcriptomics and proteomics data. The three different signatures were used as input for CMap analyses. Using a consistent enrichment score <−0.2 for all 3 approaches was applied. This resulted in 86 candidates being shortlisted. All candidates were investigated. Sixty-two of them were not further considered based on a) already described in the context of bladder cancer b) not available to be purchased or c) being toxic, carcinogen or genotoxic. The remaining 24 compounds were further investigated in in vitro systems (bladder cancer cell lines).

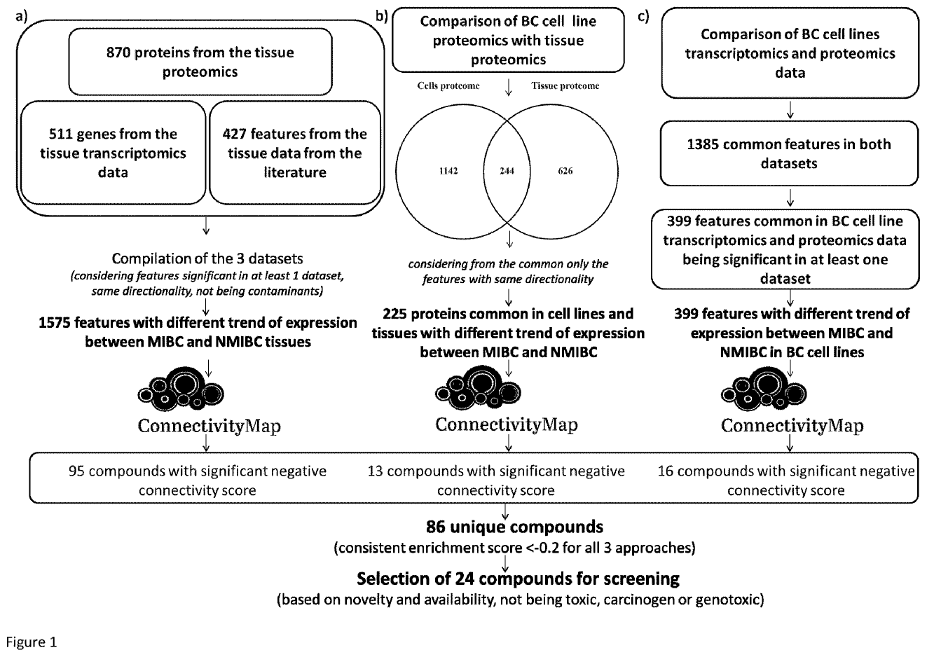

FIG. 2. The dose response curves of cephaeline (i-ii), exemestane (iii-iv) and 1,4 chrysenequinone (v-vi) in the BC cell lines RT112, T24, HT1376 and BFTC905. The dose response curves of fluvoxamine (vii), amiodarone (viii), isradipine (ix) and amodiaquine (x) in the RT112 cells are also provided. All of the drugs significantly decreased the proliferation rate of the BC cells in vitro.

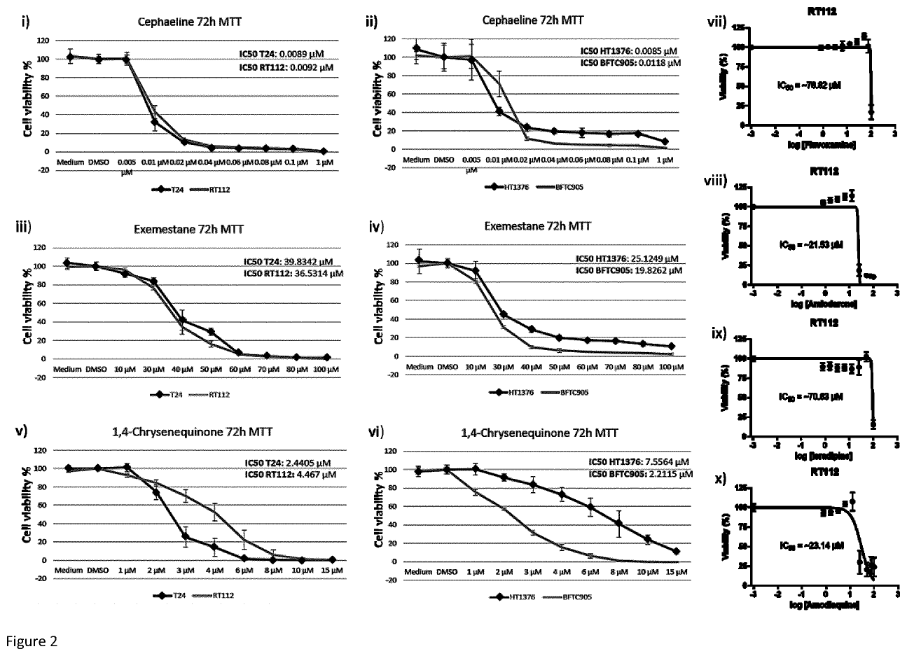

FIG. 3. The impact of cephaeline, exemestane and 1,4 chrysenequinone in vivo in BFTC905 xenograft mouse models. Cephaeline and exemestane significantly decreased tumor establishment or growth in vivo whereas no significant impact on tumour growth was observed for 1,4 chrysenequinone.

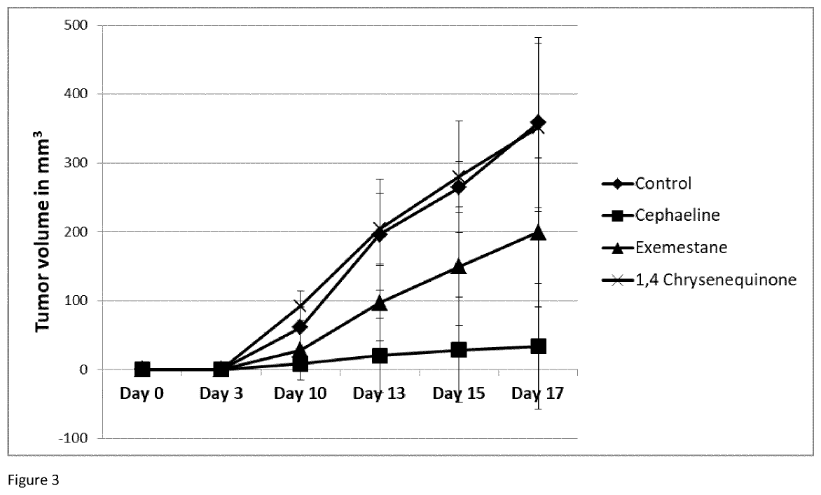

1. A method for determining candidate molecules for treating a disease, disorder or condition associated with tissue alteration, like a tumor, in particular bladder cancer (BC) at different stages of said disease, disorder or condition comprising in silico prediction of drug response, based on reversion of a disease specific molecular signature, in particular, applying CMap analysis based on data obtained by
i) a first method of obtaining a disease, disorder or condition specific signature, like BC signatures from the altered tissue, like from BC tissue whereby a) these tissues are analyzed on proteomic differences associated with disease, disorder or condition associated progression, like tumor progression, in particular with BC progression and be detected in more than 10% of the analyzed tissue samples followed by b) identifying features characterizing different protein expression associated with the progression or the different stages of the disease, disorder or condition for providing a first data set for in silico prediction of drug response, in particular by CMap analysis;
ii) a second method of obtaining a disease, disorder or condition specific signature, like BC signatures whereby a) the cell proteome of cell lines, like tumor cell lines, in particular, BC cell lines depicting MIBC and NMIBC pathology, of different stages of progression of said disease, disorder or condition are analyzed and determining proteins demonstrating significant change in abundance between different stages of progression followed by b) comparing the protein of the cells, like the tumor cells, in particular the BC, with the tissue proteome for providing a second data set for in silico prediction of drug response, in particular by CMap analysis;
iii) a third method of obtaining a third disease, disorder or condition specific signature, like a third BC signature comprising a) obtaining a cell lines transcriptomics data set whereby cell lines of different stages of the disease, disorder or condition are analysed; b) obtaining a cell line proteomics data set whereby cell lines of different stages of the disease, disorder or condition are analysed; c) comparing the proteomics data and transcriptomic data of a) and b) for obtaining features common in both data sets; and d) from the common features obtained in c) determining features being statistically significant different in at least one of the data set of a) and b) for providing a third data set for in silico prediction of drug response, in particular by CMap analysis; and
iv) the data set obtained in each of i), ii) and iii) are analyzed by a method for in silico prediction of drug response, in particular by CMap analysis for determining candidate molecules.

2. The method for determining candidate molecules for treating a disease, disorder or condition, associated with tissue alteration, like a tumor, in particular BC according to claim 1 wherein the disease is a tumor including solid tumors.

3. The method according to claim 2 wherein the tumor is BC.

4. The method according to claim 3, wherein BC progression is differentiated between non-muscle invasive bladder cancer (NMIBC) and muscle invasive bladder cancer (MIBC).

5. The method according to claim 2 wherein the tumor is prostate cancer.

6. The method according to claim 1 wherein the disease, disorder or condition is heart failure.

7. The method for determining candidate molecules for treating a disease, disorder or condition, associated with tissue alteration, like a tumor, in particular BC according to claim 1 wherein the candidate molecules are determined to allow reversal of an aggressive phenotype of the disease, disorder or condition, associated with tissue alteration, like a tumor, in particular BC.

8. The method for determining candidate molecules for treating a disease, disorder or condition, associated with tissue alteration, like a tumor, in particular BC according to claim 1 wherein in step i) after b) the features of b) are compared with tissue transcriptomics data and/or features from tissue databases for providing features differentially expressed at the different stages of said disease, disorder or condition, like being relevant to stage progression, in particular, BC progression present in the first data set.

9. The method for determining candidate molecules for treating a disease, disorder or condition, associated with tissue alteration, like a tumor, in particular BC according to claim 8 wherein the cell lines analyzed in method ii) are cell lines having either MIBC or NMIBC phenotype.

10. The method for determining candidate molecules for treating a disease, disorder or condition, associated with tissue alteration, like a tumor, in particular BC according to claim 1 wherein proteins determined in step ii) included among others proteins for which disease specificity in the analysed tissue, like tumor association, is known.

11. The method for determining candidate molecules for treating a disease, disorder or condition, associated with tissue alteration, like a tumor, in particular BC according to claim 1 wherein the tissue proteome used in step ii) is the tissue proteome of step i).

12. The method for determining candidate molecules for treating BC according to claim 1 wherein the transcriptome in step iii) is RNA transcriptome from cell lines from urinary tract.

13. The method for determining candidate molecules for treating a disease, disorder or condition, associated with tissue alteration, like a tumor, in particular BC according to claim 1 wherein in step iv) candidate molecules are determined by a negative enrichment score in CMap analysis.

14. The method for determining candidate molecules according to claim 1 further comprising the step of determining data on treating the disease, disorder or condition, associated with tissue alteration of said candidate molecules determined in the present method in treating a different disease than the disease of claim 1.

15. Method for determining candidate molecules according to claim 14, wherein the step of determining data on treating a disease is a disease being a tumor and wherein the candidate molecules are known candidate molecules in treating cancer whereby said cancer is a different cancer disease different to the cancer disease of claim 1.

16. The method for determining candidate molecules according to claim 15 wherein the disease is BC and the data determined on treating a disease are data determined with respect to a cancer other than BC and the candidate molecules are known candidate molecules in treating cancer other than BC.

17. A candidate molecule not described for treating a disease, disorder or condition associated with tissue alteration, like a tumor, in particular BC at different stages of said disease, disorder or condition obtained by a method according to claim 1 for use in treating a disease, disorder or condition associated with tissue alteration, like a tumor, in particular BC at different stages of said disease, disorder or condition.

18. The candidate molecule according to claim 17 for use in the treatment of BC being Cephaeline.

19. The drug candidate according to claim 17 being a compound of general formula II 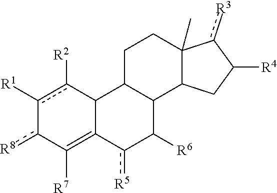
wherein
R₁, R₂, R₄, R₆ and R₇ each are independently selected from H, C₁-C₄ alkyl, substituted C₁-C₄ alkyl, halogen;
R₃ and R₈ are each individually selected from O, OH, O C₁-C₄ alkyl;
----- is a single or double bond;
R5 is a compound of CHn (C1-C4) m with n and m being an integer of 0 to 3 and n+m is 3 when ----- is a single bond, and
R5 is CH2, CH C1-C4 alkyl or C(C1-C4 alkyl)2 when ----- is a double bond,
for use in the treatment of BC.

20. The candidate molecule of general formula II for use in the treatment of BC according to claim 19 wherein R₁, R₂, R₄, R₆ and R₇ are hydrogen; R₃ and R₈ are O and R₅ is CH₂
----- is a double bond, in particular, is exemestane.

21. The candidate molecule according to claim 19 for use in treating of BC being a compound selected from the group of
v) Fluvoxamine or a derivative thereof;
vi) Amiodarone or a derivative thereof;
vii) Isradipine or a derivative thereof; or
viii) Amodiaquine or a derivative thereof.
